(19) United States
(12) Patent Application Publication
(10) Pub. No.: US 2021/0346426 A1
(43) Pub. Date: Nov. 11, 2021

(54) COMPOSITION AND METHOD FOR THE TREATMENT OF COVID-19

(71) Applicants: Naeem Uddin, KARACHI (PK); Shamail Zia, KARACHI (PK)

(72) Inventors: Naeem Uddin, KARACHI (PK); Shamail Zia, KARACHI (PK)

(21) Appl. No.: 17/225,053

(22) Filed: Apr. 7, 2021

Related U.S. Application Data
(60) Provisional application No. 63/006,720, filed on Apr. 8, 2020.

(51) Int. Cl. A61K 33/30; A61K 9/00; A61K 9/48; A61K 33/00; A61K 31/194; A61P 31/14

(52) U.S. Cl. CPC A61K 33/30; A61K 9/0053; A61P 31/14; A61K 33/00; A61K 31/194; A61K 9/4808

(57) ABSTRACT

A composition and method for treating COVID 19 by orally administering a pharmacological composition comprising a pharmaceutical carrier and an active composition consisting of about 82% to about 92% Sodium citrate, about 8% to about 18% citric acid and an effective amount of zinc sulfate.

### RELATED APPLICATIONS

This application claims priority of provisional applications 63/006.720 filed on Apr. 8, 2020. The entire disclosure of this application is incorporated herein by reference.

### SPECIFICATION

### Field of the Invention

The composition of this invention is an anti-viral composition for the treatment and management of COVID-19 in patients having such infections. The composition comprises a mixture of sodium citrate and citric acid, preferably in a stoichiometric ratio, and an amount of zinc sulphate.

### Background

Due to the worldwide pandemic due to the SARS-CoV-2 there is a dire need for new antiviral compositions for the management of the outbreak of Coronavirus or COVID-19 At the date of filing this application, as reported to WHO (https://covid19.who.int/), globally there have been over 2.8 Million deaths and 131 Million confirmed cases of COVID-19 and over 550,000 deaths in the United States. Social distancing is a major way to mitigate the transmission of COVID-19

Social distancing is a major way to mitigate the transmission of COVID-19. Additionally, teaching about the proper way to cough, maintain hygiene and social distancing is important. Not following these techniques is a significant cause of COVID-19 spreading throughout the world.

When the people from the Wuhan City of Hubei province in China, where COVID-19 first broke out, traveled to countries like Europe and United States, COVID-19 started spreading and infecting the world. Miraculously, several vaccines have emerges to mitigate and/or prevent COVID-19 infecting the global population.

The Coronavirus is named after the crown like spikes that protrude from the virus's surface. The virus is believed to have originated from bats and Pangolins. This virus is wrapped around the bubble of oily lipid molecules. It enters the body through the nose, mouth or eyes, then attaches to cells in the airway that produce a protein call ACE2. The virus infects the cell by fusing it's spikes with the membrane of the cells via ACE2 protein. After fusing to the membrane of the cells it enters into the cell in the shape of a single messenger RNA. Then inside cell, the cells start reading the messenger RNA as its own and starts making more and more copies of this RNA. This virus keeps the immune system silent as it takes over the machinery of the cell. Not only is RNA formed but the spike protein starts making new virus RNA inside the cells and then the cells rupture and countless virus with their spike protein shell enters into the blood infecting more cells of the body.

### Citric Acid

Discovery: Citric acid was first isolated in 1784 by the chemist Wilhelm Scheele, who crystallized it from lemon juice. Industrial-scale citric acid production first began in 1890. In 1893, Carl Wehmer discovered that penicillium mold could produce citric acid. In 1917, an American food chemist James Currie discovered certain strains of the mold Aspergillus niger could efficiently produce citric acid.

Citric Acid, 2-hydroxytricarboxylic acid, because of its biological origin, its functionality makes it suitable for a wide range of applications. The presence of one hydroxyl group and 3 carboxyl groups permits the formation of complex molecules, which may be soluble and capable of modifying the solubility of a constituent's material. Citric acids, Oxalic acid, along with lactic acid, acidulent and its salts are typically buffered in pharmaceutical preparations.

Citric acid crystallizes from a cold aqueous solution as monohydrate (C₆H₈O₇H₂O); HOC(COOH)(CH₂COOH)₂.H₂O

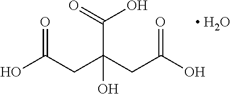 [SMILES: O=C(O)CC(O)(CC(=O)O)C(=O)O ; "· H2O"]

PROPERTIES CITRIC ACID
NAMES
IUPAC name
Citric acid
Systematic IUPAC name
2-Hydroxypropane-1,2,3-tricarboxylic acid
IDENTIFIERS

| | |
|---|---|
| CAS Number | 77-92-9 |
| Chemical formula | C₆H₈O₇ |
| Molar mass | 192.123 g/mol (anhydrous), |
| | 210.14 g/mol (monohydrate) |
| Appearance | white solid |
| Odor | Odorless |
| Density | 1.665 g/cm³ (anhydrous) |
| | 1.542 g/cm³ (18° C., monohydrate) |
| Melting point | 156° C. (313° F.; 429 K) |
| Boiling point | 310° C. (590° F.; 583 K) |
| | decomposes from 175° C. |
| Solubility in water | 54% w/w (10° C.) |
| | 59.2% w/w (20° C.) |
| | 64.3% w/w (30° C.) |
| | 68.6% w/w (40° C.) |
| | 70.9% w/w (50° C.) |
| | 73.5% w/w (60° C.) |
| | 76.2% w/w (70° C.) |
| | 78.8% w/w (80° C.) |
| | 81.4% w/w (90° C.) |
| | 84% w/w (100° C.) |
| Solubility | Soluble |
| | in acetone, alcohol, ether, ethyl |
| | acetate, DMSO |
| | Insoluble in |
| | C₆H₆, CHCl₃, CS₂, toluene |
| Solubility in ethanol | 62 g/100 g (25° C.) |
| Solubility in amyl acetate | 4.41 g/100 g (25° C.) |
| Solubility in diethyl ether | 1.05 g/100 g (25° C.) |
| Solubility in 1,4-Dioxane | 35.9 g/100 g (25° C.) |
| log P | −1.64 |
| Acidity (pKₐ) | pKₐ₁ = 3.13 |
| | pKₐ₂ = 4.76 |
| | pKₐ₃ = 6.39,^{[6]} 6.40 |
| Refractive index (n_{D}) | 1.493-1.509 (20° C.) |
| | 1.46 (150° C.) |
| Viscosity | 6.5 cP (50% aq. sol.) |
| Crystal structure | Monoclinic |
| Heat capacity (C) | 226.51 J/(mol · K) (26.85° C.) |
| Std molar entropy (S^{⊖}₂₉₈) | 252.1 J/(mol · K) |
| Std enthalpy of formation (Δ_{f}H⊖₂₉₈) | −1543.8 kJ/mol |
| Heat of combustion, | 1985.3 kJ/mol (474.5 kcal/mol, |
| higher value (HHV) | 2.47 kcal/g),^{[4]} 1960.6 kJ/mol |
| | 1972.34 kJ/mol (471.4 kcal/mol, |
| | 2.24 kcal/g) (monohydrate) |
| ATC code | A09AB04 (WHO) |

The crystal is colorless. It is optically inactive. Citric acid is an organic acid as indicated by the first dissociation constant which is 8.2×10⁻⁴. The second and third dissociation constants are 1.77×10⁻⁴ and 3.9×10⁻⁷ respectively. Citric acid is readily soluble in water and in varies organic compound.

It is a natural preservative present in citrus fruits. It is white hygroscopic crystalline powder. It can exist either in an anhydrous (water-free) form or as monohydrate citric acid also dissolves in absolute (anhydrous) ethanol Citric acid is also used to add an acidic or sour taste to foods and drinks. It is used mainly as an acidifier, a flavoring and chelating agent.

The US FDA lists citric acid in the Nov. 20, 1959 issue of the Federal Register (23-a) as a substance “generally recognized as safe” for specific use in compliance with the Food Additive Amendment of 1958 Even at high concentration, citric acid is not injurious in contact with skin.

Oral Administration: After oral administration the citrate ion is rapidly and almost completely oxidized with less than 1% being excreted unchanged in urine. Intravenous injection shortens, coagulation times of the blood but in vitro, the citrate ion acts as an anticoagulant.

With sodium bicarbonate, citric acid is used in many effervescent powder and tablet to liberate carbon dioxide in addition of water. Citric acid salt's such as sodium citrate and potassium citrate and citric acid are also used in different remedies, such as Citro Soda® (Abbott Laboratories).

### Sodium Citrate

Sodium Citrate in conjunction with citric acid is used as buffering agent in preparation of candies for controlling PH. In pharmaceutical preparation such as effervescent tablets, powders and droughts, sodium citrate is used as blood and urinary alkalizer and in large dosage as saline cathartic. Due to anticoagulant property of citrate ion, sodium citrate is extensively employed for this purpose and the final product is known officially in US Pharmacopeia as citrated, normal human plaza. When Blood is drawn from an individual under aseptic condition into sterile bottle, the bottle contains 50 ml of a 4% solution of sodium citrate in isotonic sodium chloride solution. To this is added 50 ml of whole blood. The sodium citrate prevents the blood from coagulation.

### Zinc Sulphate:

| ZnSO4 | Zinc Sulfate | |
|---|---|---|
| Molecular Weight/Molar Mass | 161.47 | g/mol |
| Density | 3.54 | g/cm³ |
| Boiling Point | 740° | C. |
| Melting Point | 680° | C. |

Zinc sulfate appears as colorless or white rhombic crystals or powder at room temperature. It has convergence property and is easily soluble in water with its aqueous solution being acidic. It is slightly soluble in ethanol and glycerol. Pure zinc sulfate can be stored in the air for a long time without turning yellow. It can lose water to become white powder when placed in dry air. At the range of 60-100° C., it will become zinc sulfate monohydrate. When heated to 280° C., it will completely lose water, decompose into zinc sulfite at 680° C. and further decompose at above 750° C. and finally decomposition into zinc oxide and sulfur trioxide at about 930° C. ZnSO4.7H2O can form mixed crystal with MSO4.7H2O (M=Mg, Fe, Mn, Co, Ni) within a certain range.

Zinc sulfate is mainly used for the preparation of raw materials of pigment lithopone, zinc barium and other zinc compounds. It also has various kinds of applications such as animal nutrition due to a zinc deficiency, animal feed additives, crop zinc fertilizer (trace element fertilizer), important materials of artificial fiber, electrolyte solution upon electrolytic production of zinc metal mordant m the textile industry, pharmaceutical emetic agents, astringents; fungicides and wood and leather preservatives. It can be derived from the reaction between zinc or zinc oxide and sulfuric acid or from the baking of sphalerite in the baking furnace followed by extraction and refining.

The anhydrous sulfate is a colorless rhombohedral crystalline solid; refractive index 1.658; density 3.54 g/cm³; decomposes at 600° C., soluble in water, methanol, and glycerol. The heptahydrate, ZnSO₄+7H₂O, is a colorless crystalline solid; metallic taste; rhombohedral crystals; effloresces; refractive index 1.457, density 1.957 g/cm³ at 25° C.; melts at 100° C.; loses all its water molecules at 280° C.; decomposes above 500° C.; very soluble in water, 96.5 g/100 mL at 20° C.; soluble in glycerol, 40 g/100 int; insoluble in alcohol. The hexahydrate, ZnSO₄?6H₂O constitutes colorless monoclinic or tetragonal crystals; density 2.072 g/cm³ at 15° C.; loses five water molecules at 70° C.; soluble in water.

References relating to these ingredients which are incorporated herein by reference are.

U.S. Pat. No. 9,301,935 to Uddin;

U.S. Pat. No. 9,962,347 to Uddin;

U.S. Pat. No. 10,493,050 to Uddin;

U.S. Pat. No. 10,646,461 to Uddin;

### SUMMARY OF INVENTION

This invention is directed to an orally administered pharmacological composition for administration to a patient orally for the treatment of COVID-19. The composition comprises a pharmaceutical carrier and an active composition consisting of about 82% to about 92% Sodium citrate, about 8% to about 18% citric acid and an effective amount of zinc sulfate Preferably; the active composition consists of about 82% to about 92% Sodium citrate, about 8% to about 18% citric acid and an effective amount of zinc sulfate, and wherein the pharmacological composition contains from about 300 mg to about 800 mg of sodium citrate and citric acid, and about 10 to 40 mg of zinc sulfate as an effective amount. The highly preferred active composition includes about 500 mg of sodium citrate and citric acid and about 20 mg zinc sulfate. Preferred, is an active composition consisting of about 87% Sodium citrate, about 13% citric acid and an effective amount of zinc sulfate, i.e., 10 to 40 mg, and is an encapsulated pharmacological composition. The pharmacological composition is taken at least twice a day and, if needed, three times a day, over a period of at least 10 to 15 days.

### DETAILED DESCRIPTION OF INVENTION

### Preparation Active Composition

We take an equal amount of sodium bicarbonate and citric acid by weight. Stoichiometrically, Citric acid is present in larger amount in comparison to sodium bicarbonate. Small amounts of citric acid are left untreated. The final mixture contains sodium citrate and citric acid as it shown by PH of final product (the measured PH of final product is 6).

The preferred active ingredients of this invention contain sodium citrate derived from sodium bicarbonate salt and anions derived from citric acid.

An equal amount of pharmaceutical grade sodium bicarbonate and citric acid by weight was placed in a sterile plastic container in an open room with a temperature at about 25 degree Celsius. The composition was mixed until a uniform mass was formed A specific amount of purified sterile water was poured slowly in specific amount into the mass to allow the reaction of sodium bicarbonate and citric acid. The mixture was left standing for 24 hours to allow the water and carbon dioxide to evaporate and for the product to dry. The mixture was again stirred to allow carbon dioxide and water vapors to further escape. A white crystalline and odorless powder was obtained. The final product contained 87% sodium Citrate and 13% citric acid. The measured pH of final product was 6 This solid composition was crushed and ground to a fine powder to produce a composition that was pharmaceutically and physiologically acceptable, herein in the “pharmacological composition”

### Sodium Citrate—Citric Acid Mixture

### Stoichiometric Formulation Ratio:

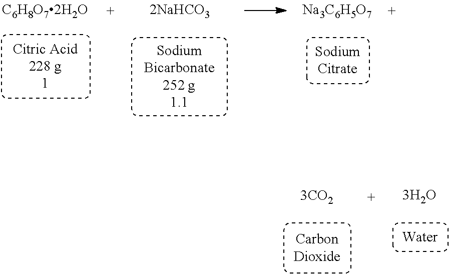

### Method of Producing Active Composition:

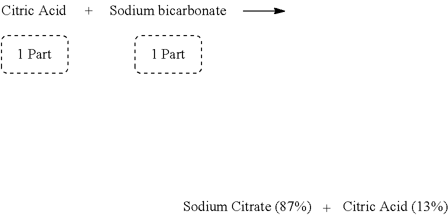

Chemical Equation with Structural Formulas:

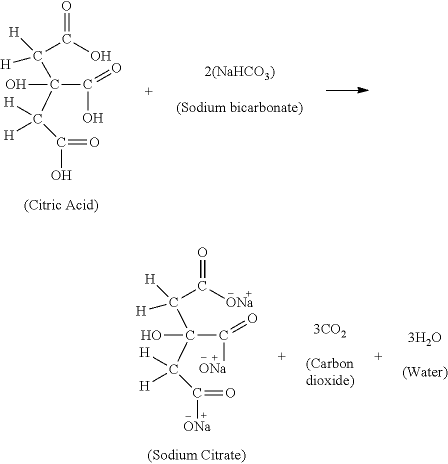

### Structural Formulae:

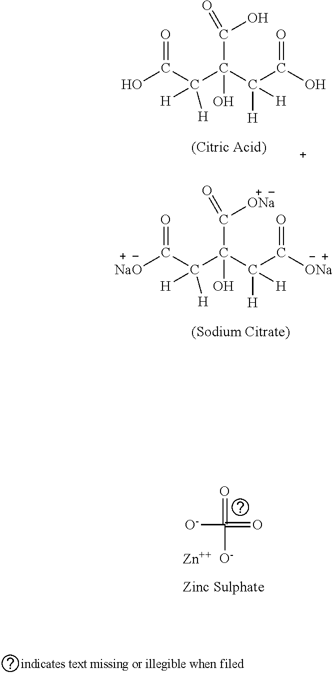

The theoretical stoichiometric ratio of citric acid and sodium bicarbonate for the preparation of mixture of sodium citrate and citric acid is 1:1.2 weight ratios. The composition used in the pharmacological compositions of this invention uses a 1:1 weight ratio of citric acid to sodium bicarbonate to make the composition (SCCA) This produces a final composition (SCCA) with an excess of citric acid mixed with sodium citrate. This imparts a safe and efficacious activity to the composition. The composition maintains a pH of about 6 which is acceptable for patient use.

The highly preferred pharmacological composition contains a mixture of 87% sodium citrate and 13% citric acid (SCCA). A preferred range is about 82% to about 92% Sodium citrate and about 8% to about 18% citric acid. It is to be understood however that various weights and ratios of sodium citrate and citric acid may be used as long as a safe and efficacious pharmacological composition is produced.

Sodium Citrate—Citric Acid Mixture

### a) Stoichiometric Formulation Ratio:

$$\begin{matrix}C 6 H 8 O 7.2 H 2 O + 3 NaHCO 3 & C 6 H 5 O 7 Na 3 + 2 CO 2 + 2 H 2 O \\ \begin{matrix}( Citric acid ) & + & ( Sodium bicarbonate ) \\ 1 Part & & 1.2 Part\end{matrix} & ( Sodium citrate )\end{matrix}$$

### b) Method of Producing SCCA Composition:

$$\begin{matrix}( Citric acid ) & + & ( Sodium bicarbonate ) \\ 1 Part & & 1.2 Part\end{matrix} \begin{matrix}Sodium citrate ( 87 \% ) + Citric acid ( 13 \% ) \\ Active composition\end{matrix}$$

### c) Addition of Zinc Sulfate to Making Final Active Composition.

$$\begin{matrix}SCCA Composition & + & ZnSo 4 \\ 500 mg & & 25 mg\end{matrix}$$

### Method of Preparation of Final Active Composition for Use:

Mix one part of citric acid and one part of sodium bicarbonate than gradually spray in it, specific quantity of sterile water sufficiently to make it complete reaction and evaporate the water and carbon dioxide completely. The water is merely the medium for the reaction. Carbon dioxide is evaporated rapidly and water is gradually evaporated. The remaining product is a mixture of 87% Sodium citrate and 13% citric acid active ingredient (“SCCA”). The product is in the form of crystals. The product is then dried and crushed to fine particles to produce an SCCA composition. Zinc Sulphate (“ZS”) is now added into the active SCCA composition and the (weight/weight) ratio of SCCA and ZS will be 500 mg of SCCA with 25 mg of ZS (Zinc Sulfate) to produce the “active treatment composition”. This composition is then encapsulated for use in the treatment of COVID-19 in patients.

Theoretical Mode of Action of the Active Treatment Composition of this Invention

It is theorized that the active treatment composition of this invention inhibits the synthesis and multiplication of COVID-19-SARS-COV-2. The active treatment composition disrupts the outer layer of Coronavirus that is made up of lipid, but does not disrupt the host cell. It is also believed that the active treatment composition disrupts the crown of Coronavirus, which is made up of protein, as the property of active treatment composition of this invention has keratolytic activity.

The active treatment composition of this invention also has anticoagulant properties which prevents intravascular clotting and prevents the formation of emboli which causes pulmonary emboli, cardiac arrest and ischemic strokes.

The active treatment composition of this invention alters the blood pH of the human body which creates an environment hostile to virus replication.

The active treatment composition of this invention is also highly effective prophylactically in adjunct therapy for the management of COVID-19 patients

The active treatment composition of this invention is also useful as a neutraceutical supplement as an adjunct to COVID-19 treatments.

### Administration of Composition

The active treatment composition of this invention may be taken orally and as a topically applied sanitizer

### Side Effects

The several side effects when taken orally are vomiting and diarrhea. The active treatment composition of this invention functions as an anti-coagulant so it may increase bleeding time.

### Clinicaltrials (In-Vivo Study)

Clinical trials for the active treatment composition of this invention were performed on 60 COVID-19 infected patients who tested positive in a COVID-19 PCR (polymerase chain reaction) test. All 60 patients were cured Below are the details on 15 of these patients.

### Patient 1

### Gender Male

Age 34 years

### Status: Accountant.

### Diagnosis: COVID-19

Present complaint: Patient complains of fever, body ache, sneezing and shortness of breath, for the last 4 days but later on he gradually felt the loss of taste and smell. He also says that one of his female colleagues has also developed loss of taste. He does not give any family history of the similar symptoms but he gives history of traveling from his home town after attending marriage ceremony where some of the persons were suffering from COVID-19.
On examination: Patient appears to be lethargic, his temperature was 99° F. Patient complains of high, low grade fever on and off, which does return to normal. His blood pressure is normal. In the light of these symptoms, the patient is advised to take a PCR for COVID-19, blood CBC (complete blood picture), chest X-ray and D-dimer test and advised to revisit after obtaining all the test results and reports, advised him to follow social distancing guidelines and to self-quarantine.

### 2^{nd} Visit:

Patient visited 2 days after the previous visit with all his tests and reports. His COVID-19 PCR test was positive, D-dimer was raised and Blood CBC report showed a disproportionate amount of lymphocytes and neutrophils. The X-ray report showed a small patch on the right side of the lung.

Rx: Patient is given active treatment composition of this invention twice daily a half hour after food for 5 days and advised to revisit after 5 days.

### 3^{rd} Visit:

Patient revisited five days after the last visit to say that he had relief and his symptoms have decreased to the extent that his taste, which had been lost, was regained. His lack of smell is somewhat improved but still persists. His fever has subsided and his breathlessness is under control to a large extent. The patient is again given the same treatment and advised to revisit after 5 days with the investigation of Blood CBC, D-dimer and x-ray chest PA view.

### 4^{th} Visit:

Patient visited 5 days after the previous visit saying that all signs and symptoms have disappeared and his temperature remains normal, his taste and of smell ability have been regained. The X-ray report shows healing of the patch of the lung and there was no patch infection in the lungs, which indicated that the effect of virus ceased as there was no lesions developed and D-dimer report was improved. His course of treatment was stopped after 10 days of treatment with the active treatment composition of this invention. The patient is advised to consume a proper diet and rest and requested to return in 20 days.

### 5^{th} Visit:

The patient revisited 12 days after the previous visit with the reports of PCR COVID-19, Blood CBC, D-dimer and chest X-ray PA view. All tests were normal. The patient was advised to remain away from crowds, particularly those suffering from COVID-19, as he may be re-infected if he has low immunity.

### Patient 2

Age 58 years

### Gender: Male

### Diagnosis: COVID-19

Status: Assistant Supervisor in Private firm
Present complaints: Patient complained of on and off fever and also complained of, breathlessness, cough and sneezing for the last 6 days. He was getting treatment from nearest doctor but he did not get relief at all.
On Examination: His fever is 101° F., pulse 106 bpm, BP normal, on auscultation there was respiratory distress. Patient is advised to get tests including PCR for COVID-19, Blood CBC, D-dimer and chest x-ray PA view and visit after getting all recommended investigations.

### 2n^{d} Visit:

Patient visited 2 days after previous visit, the report showed PCR positive for COVID-19, D-dimer was normal, Blood CBC shows disproportionate count of lymphocytes, neutrophils and declined platelet counts. X-ray report showed marked infection as big patch on lungs.

Rx: Patient was given active treatment composition of this invention encapsulated, twice a day for 5 days and advised to revisit after 5 days.

### 3^{rd} Visit:

Patient revisited 5 days after previous visit to say that he has obtained substantial relief, his fever has subsided and he has substantial relief from his breathlessness. He is feeling very well compared too previously, but still he sometimes has breathlessness and a body ache. X-ray report shows no spread of infection indicating that drug is affecting on patient. Patient is given same remedy as previously and advised to revisit after 5 days

### 4th Visit:

Patient revisited 5 days after previous visit. Patient is very satisfied and says that now he has a lot of relief There is no breathlessness. Since the patient's signs and symptoms have declined, but some of the problem like breathlessness sometimes occurs. X-ray report does not show any patch of infection and is also showing the healing of lesion. An additional 2 days more of treatment is recommended. The patient was advised to do the PCR for COVID-19 after 20 days from the day of starting medicine.

### 5^{th} Visit:

Patient visited 14 days after previous visit where all his reports had become normal as well as PCR for COVID-19 became negative-ray report also showed improvement

### Patient 3

Age: 52 years
Gender: male
Status: Assistant professor of chemistry

### Diagnosis: COVID-19

Present Complaints: Patient complains of severe cough, sneezing, irritation in throat, shortness of breath, hoarseness of voice and with intermittent fever ranging from 99° F. to 103° F. High grade fever, dry cough, sneezing and body ache. Family history is the death of his elder brother in COVED-19 four days before. He used to go daily to his elder brothers home when he was sick. Upon Examination: Patient appears sick, lethargic and weak. Temperature 100° F., pulse 98 bpm. On auscultation of chest crepts, a grating or crackling sound or sensation, are also found. Patient was advised to take tests for PCR for COVID-19, blood CBC, D-dimer, Blood culture and sensitivity and chest X-ray PA view as baseline investigation. Patient was advised to come just after getting basic reports, except blood culture as it will be obtained after 3-4 days.

### 2^{nd} Visit:

Patient revisited 2 days after 1st visit with test reports except blood culture and sensitivity. The report shows 1) PCR for COVID-19 positive, 2) CBC report shows disproportion of neutrophils and lymphocytes, 3) chest x-ray shows big patch of infection, 4) D-dimer raised.

Rx: Patient is given encapsulated active treatment composition to take twice a day half an hour after food, advised to remain in quarantine and revisit after 5 days.

### 3^{rd} Visit:

Patient revisited 5 days after previous visit. He stated that his fever subsided, cough, irritation in throat, breathlessness and hoarseness of voice reduced in severity and he has obtained some relief Patient is given same encapsulated active treatment composition for 5 days of treatment and advised to revisit within 5 days.

### 4^{th} Visit:

Patient revisited 5 days after his previous visit. He stated that he has limited relief in his condition, but he still complained of a cough and a negligible persistent irritation in his throat. The patient was given the same encapsulated active treatment composition for 3 more days with the advice to come with blood CBC, D-dimer chest X-ray tests after 3 days.

### 5^{th} Visit:

Patient revisited 3 days after previous visit as per advice to say that he has complete relief, no fever, throat infection, irritation, his report shows normal blood CBC, D-dimer declined and X-ray shows healed lung lesions and there was no furthermore extension in lungs infection. After overall examination of patient and review of the tests, it showed that patient is now cured. However, he was given the same medicine for 2 days and advised him to do the test of PCR COVID-19 5 days after the completion of 15 days medicine and revisit with the PCR COVID-19 report.

### 6^{th} Visit:

Patient visited 7 days after previous visit with negative PCR report for COVID-19 and improvement of the chest x-ray and there was no further infection in lungs and the rest of the reports were normal.

### Patient 4

Gender: female
Age: 26 years

### Diagnosis: COVID-19

Family Status: Her family members had travelled abroad and they got COVID-19 and were under the treatment.
Present Complaint: She also has developed the same sign and symptoms like her family members including breathlessness, fever, body ache, cough, flu like condition and sneezing Upon examination of Patient having fever 99° F., Pulse 95 bpm and myalgia. Patient does not appear to be lethargic, patient is advised to do the PCR for COVID-19 which was positive and the treatment for COVID-19 is started. Patient is started on treatment with the encapsulated active treatment composition for twice a day for 5 days and advised revisit after 5 days.

### 2nd Visit:

Patient visited 5 days after the previous visit to say that she has got relief and her signs and symptoms have declined and even fever has subsided but myalgia and slight cough still persist. Patient is given the same remedy for further 5 days more Upon examination of the patient she was completely cured and there was no fever, myalgia and cough. Patient was again given the same remedy for further 5 days.

### 3^{rd} Visit:

Patient visited 5 days after previous visit, Upon examination patient appears to be completely cured without having fever, cough and myalgia. She was again given the same remedy for further 5 days to be on the safe side and advised to revisit after 10 days with the report of PCR COVID-19.

### 4^{th} Visit:

Patient visited 10 days after the previous visit with negative PCR COVID report. Patient treatment was stopped.

### Patient 5

Gender: male
Age: 70 years
Status: Married, 4 sons, 1 daughter
Profession: Owner of Primary care hospital

### Diagnosis: COVID-19

Present Complaints: Patient complained of fever, running between 99.5 to 101° F., dry cough, running nose, body ache & shortness of breath for the last 3 days. Patient has Diabetes mellitus and had undergone a Cardiac bypass. Patient was advised to visit the cardiologist to exclude symptoms of breathlessness from a heart condition.

### 2^{nd} Visit:

The next day after 1st visit, patient came with prescription of medicine which he is taking and also the advice of cardiologist. Cardiologist excluded the case of breathlessness due to cardiac problems and said it is due to some other cause. Upon examination during the 2^{nd} Visit the fever was 100° F., Bp140/90, pulse 82 bpm. Crepts on chest auscultation.

Investigation: Patient is PCR for COVID-19 positive
Rx: Started with active encapsulated treatment composition for 5 days and advised to revisit after 5 days.

### 2^{nd} Visit:

Patient revisited 5 days after the previous visit to say that he has relief and all his symptoms have subsided up to an extent. Patient is given the same remedy and advised to revisit after 5 days.

### 3^{rd} Visit:

Patient revisited 5 days after the previous visit with complete satisfaction and says that his breathlessness has subsided. Patient is given the same remedy for further 5 days and advised to revisit after 10 days for an examination.

### 4th Visit:

Patient revisited 10 days after the previous visit with complete satisfaction and cure of all his symptoms and on examination there was no any signs of complication of disease. Treatment is stopped.

### Patient 6

### Gender: Male

### Age: 65

### Status: Married

Profession: Works in Primary care Hospital

### Diagnosis: COVID-19

Present complaints: Patient complains of fever, body ache, suffocation and shortness of breath for the last 5 days but at the time of his first visit he had severe breathlessness and cough. When he was talking he was unable to talk. He is a known case of cardiac problem and he underwent stent operation for his cardiac problem. He was advised to get advice from cardiologist to exclude the symptoms due to cardiac problem. He called cardiologist to check him and advice. Cardiologist excluded symptoms due to cardiac problem.
Examination: fever 99° F., chest congested, throat inflamed.
Investigation: Patient was advised for COVID-19 PCR test and X-ray, chest PA view immediately, x-ray showed haziness, and pneumonic patches and PCR was positive.
RX: Started the active encapsulated treatment composition for 5 days advised to revisit after 5 days

### 2^{nd} Visit:

Patient revisited 5 days after the 1st visit and as per his statement patient got some relief and his sign and symptoms are subsided up to and extent. Upon examination patient seemed to be better than before. Patient was advised to continue the same active encapsulated treatment composition for further 5 days and revisit after 5 days.

### 3^{rd} Visit:

Patient revisited after the 5 days of previous visit. Patient condition appears to be improved. Patient says that he has revived to a great extent. His fever subsided, breathlessness declined, and the cough also declined. Upon examination the patient's fever had subsided and on auscultation his crepts and congestion are subsided and patient appears to be 80% better than his 1^{st} Visit condition. Patient was given the same remedy for further 5 days. Patient was advised to revisit after 5 days.

### 4^{th} Visit:

Patient revisited 5 days after the previous visit and said that he was almost cured. All his signs and symptoms of breathlesness, cough and congestion have subsided. Upon examination of the patient he was almost cured, but even though his condition was very severe he was given 5 more days of treatment with the active encapsulated treatment and also advised to revisit after 5 days. Advised for PCR test and bring report in next visit.

### 5^{th} Visit:

Patient revisited 5 days after the previous visit. Patient statement and examination of the patient shows that he is almost completely cured and there is no need for further treatment. Treatment was stopped. PCR COVID report negative.

### Patient 7

Age: 40 years
Gender: male

### Status: Married

Profession: Medical Director of primary care hospital
Present Complaints: Patient complained of fever, body ache, breathlesness and cough for last 2 days. His father and Uncle were positive for COVID-19 PCR
Upon Examination: fever 99° F., pulse 82 bpm, 170/100 B.P.
Investigation: Patient already had his COVID-19 PCR done and it was positive, chest x-ray, cbc reports were also available and chest x ray shows patch on left lower lobe, cbc report shows dispropotianate lymphocyte and neutrophils. Patient was started active encapsulated treatment composition to take orally twice a day and advised to revisit after 5 days.

### 2^{nd} Visit:

Patient revisited after the 5 days of previous visit and patient stated that he had relief to an extent and upon examination, signs and symptoms were also declining Same treatment continued and patient was advised to revisit after 5 days.

### 3^{rd} Visit:

Patient revisited 5 days after the previous visit. Patient was completely satisfied and said that all his symptoms had subsided. Upon examination patients signs of fever, chest infections, and cough had subsided. Treatment was stopped and patient was advised to revisit after 10 days with report of PCR COVID-19 and chest x-ray

### 4th Visit:

Patient visited 10 days after the previous visit with the report of PCR for COVID-19 and the was negative and also in chest x-ray there was no spread of infection, which indicated that patient was cured and no need for any treatment for furthermore

### Patient 8

### Gender: Male

### Age: 38

### Status Married

Profession: practicing physician
Present complaints: Patient complained of fever, body ache and sore throat for the last 4 days.
Upon Examination: Patient fever is 98.8° F., pulse 76 bpm, BP 130/80 Chest examination showed no congestion. Throat was inflamed
Investigation: Chest X-ray, Blood CBC normal. Patient was advised for COVID-19 PCR test which was positive.
Family History: patient father and brother were also PCR for COVID-19 positive.
RX: Started the active encapsulated treatment composition for 5 days and advised to revisit after 5 days.

### 2^{nd} Visit:

Patient visited 5 days after the previous visit, Patient symptoms and signs showed that patient obtained relief to a great extent. Patient repeated the same medicine for further 5 days.

### 3^{rd} Visit.

Patient revisited 5 days after the previous visit and per patient statement he had complete relief and he doesn't need any further treatment even though he was given the medicine for an additional 3 days and advised to revisit after 10 days with the report of COVID-19 4th visit:

Patient revisited 10 days after the previous visit with negative PCR report of COVID-19

### Patient 9

Gender: female

### Age: 17

Status: Unmarried, student
Presenting complaints: Patient complains of body aches, fever, soreness of throat for last 3 days. Upon examination: fever 99° F., Pulse 77 bpm, BP120/80. Throat inflamed. Patient was advised to come back after the recommended investigation including chest x-ray blood cbc and PCR for COVID-19,

### 2^{nd} Visit.

Patient visited after 2 days of previous visit with the test reports which showed PCR for COVID-19 positive, dispropotianty of lymphocyte and neutrophil. X-ray showed patch of infection. There is family history of COVID-19 in her family. Treatment started with active encapsulated treatment composition for twice a day after meal and revisit after 5 days.

### 3^{rd} Visit:

Patient visited 5 days after the previous visit to say that soreness of throat was reduced, body aches declined but fever still persisted which was an on and off nature. Upon examination there was declination of inflammation in throat. Patient repeated the same medicine in same manner.

### 4^{th} Visit:

Patient revisited 5 days after the previous visit with satisfaction to say that his symptoms were reduced, including soreness of throat and fever which is now normal and there is no body ache. But patient seems to be weak. To be on the safe side, the patient was given the same remedy to use in same manner for an additional 3 days and come back after 10 days with the reports of PCR for COVID-19 and chest x-ray PA view.

### 5^{th} Visit:

Patient revisited 13 days after the previous visit with PCR for COVID-19 report which is negative and chest x-ray which showed the infection had subsided and there was no furthermore of the spread of infection. The patient's condition was satisfactory.

### Patient 10

### Gender: Male

Age: 18 years

### Profession: Assistant in Pharmacy Store.

Present complaints: Patient complains of loss of smell and loss of taste for the last 3 days. No history of fever, body ache, sore throat, or diarrhea. Patient was advised to get COVID-19 PCR, Blood CBC and chest x-ray and advised to come back with all the test results.

### 2^{nd} Visit:

The patient came back with all the test reports. Only PCR for COVID-19 was positive and x-ray was normal. Upon examination his temperature was normal, pulse 75 bpm, BP 120/80. Patient was given the active encapsulated treatment composition and asked to revisit after 5 days.

### 3^{rd} Visit:

The patient revisited 5 days after the previous visit to say that he had improved in taste but there was no improvement in loss of smell. Patient was given the active encapsulated treatment composition and advised to revisit after 5 days.

### 4^{th} Visit:

The patient revisited 5 days after the previous visit to say that his taste was improved and smell was, but still persisting. The patient was given the same active encapsulated treatment composition and advised to revisit after 5 days.

### 5^{th} Visit:

The patient revisited 5 days after the previous visit, just to say that his loss of taste and smell had been completely restored. The patient is advised to revisit after 5 days with test reports of PCR COVID-19 and chest x-rays.

### 6^{th} Visit

Patient revisited 7 days after the previous visit with the report of PCR for COVID-19 which was negative and there was not any infected patch or healed lesion in x-ray Treatment stopped.

### Patient 11

Age: 17 years
Gender: male
Profession: Assistant in clinic.
Presenting complaints: Fever, Bodyache, Running Nose for last 5 days.
On Examination: Fever 99° F., Pulse 88 BPM, BP 120/85 inflamed throat and sneezing. Patient is requested to come with the test reports of PCR COVID-19 blood cbc and chest x-ray.

### 2^{nd} Visit:

Patient visited with the reports of PCR COVID-19 which is positive, chest x-ray showed patch of infection and the CBC report was normal. Patient was given active encapsulated treatment composition to take orally twice a day after meal and revisit after 5 days.

### 3^{rd} Visit:

Patient revisited 5 days after the previous visit with declination of all signs and symptoms. Patient was advised to carry on the same medicine in same manner and revisit after 5 days with x ray report.

### 4^{th} Visit:

Patient revisited 5 days after the previous visit with x-ray report. Upon examination of the patient, his fever subsided, sneezing declined and the signs and symptoms of the patient showed improvement to a great extent. The patient still complained of body aches. The X-ray report showed no spread of infection but showed an improvement. The patient was given the active encapsulated treatment compositions for use for an additional 5 days and advised to revisit after 10 days with a PCR COVID-19 test report.

### 5^{th} Visit:

The patient revisited after 10 days after the previous visit with complete declination of all signs and symptoms. The X-ray report showed no spread of infection with negative COVID-19 PCR. Treatment is stopped now.

### Patient 12

Gender: male

### Age: 28

Profession: Work in Ambulance services
Status: unmarried
Presenting Complaints: Patient complained of fever, sore throat, shortness of breath for 3 days. His company did a PCR COVID-19 test from the laboratory. It was positive. Upon examination, his fever was 101° F., Pulse 88 BPM, BP 130/85, inflamed throat. The patient was advised to get a chest x-ray, blood cbc and serum ferritin.

### 2^{nd} Visit:

Patient came back with report of blood, cbc and chest x-ray. The blood cbc report showed didpropotinatey of neutrophils and lymphocytes. Serum Ferritin was raised at higher side with D-Dimer on higher side. The patient was given active encapsulated treatment composition to take orally twice a day and revisit after 5 days.

### 3^{rd} Visit:

The patient revisited 5 days after the previous visit with declination of all of his signs and symptoms. He was told to is repeat the use of active encapsulated treatment composition and advised to come back after 5 days.

### 4^{th} Visit:

The patient revisited 5 days after the previous visit with satisfactory condition and said that he had great relief. His fever subsided, soreness of throat declined, body ache is still persisting. The patient was given the active encapsulated treatment composition for treatment for the next 5 days and advised to come back after 10 days.

### 5^{th} Visit:

The patient revisited 10 days after the previous visit with negative PCR for COVID-19 and the chest x-ray showed no spread of lesion but pre-existing, chest x-ray patch was also improved. Treatment was stopped.

### Patient 13

### Age: 30

Gender: male

### Profession: Businessman

### Status: Married

Presenting complaints: Presents with slight soreness in throat and loss of smell and taste. Patient came with positive PCR report for COVID-19 report as he had already done this investigation himself Upon examination—fever 100° F. pulse 88 BPM, BP 130/85. No sore throat. Patient was given the active encapsulated treatment composition to take orally twice a day after a meal and advised to revisit after 5 days.

### 2^{nd} Visit:

The patient revisited 5 days after the previous visit. Upon examination his fever had declined and inflammation of throat had also declined. Patient said that his loss of taste had also improved. He was given the same remedy and advised him to use in same previous manner and advised him to revisit after 5 days.

### 3^{rd} Visit:

Patient revisited after 5 days with to say that his taste had returned and his soreness of throat also declined. He was given the same remedy and advised to revisit after 10 days with reports of PCR for COVID-19 and chest x-ray.

### 4^{th} Visit:

The patient revisited 10 days after the previous visit with full satisfactory condition and his PCR for COVID-19 became negative and in chest x-ray there was no any patch of infection. Treatment stopped.

### Patient 14

### Age: 24

Gender: female
Status: Married and House wife

Presenting complaints: Presents with slight soreness in throat and loss of smell and taste. Patient came with positive PCR report for COVID-19 report that she had already done this investigation. Upon examination: no fever, no high BP, only throat is inflamed. Patient was given active encapsulated treatment composition to take orally, twice a day after a meal and advised for revisit after 5 days.

### 2^{nd} Visit:

The patient revisited five days after the previous visit with gross improvement. She also said that her smell and taste improved. Same remedy is repeated in same manner and advice to come after five days.

### 3^{rd} Visit:

Patient revisited 5 days after the previous visit with great improvement. The patient's smell and taste improved 80%. Soreness of the throat declined. Patient repeated the same treatment for an additional 5 days and advised to revisit after 10 days with the test report of PCR COVID-19

### 4^{th} Visit:

The patient revisited 10 days after the previous visit with complete improvement at all ends including return of smell and taste and as per stated by the patient, soreness of throat completely subsided. Treatment was stopped.

### Patient 15

### Age: 23

### Gender: Male

Status: Student, unmarried
Presenting complaints: Patient complains of sudden fever on and off, sore throat, cough, shortness of breath for last 2 days, patient also complained of lethargy. Patient came with the positive report of PCR of COVID-19. Upon examination: Temperature was 100° F., pulse 90 BPM, BP 130/80, throat inflamed Patient was given the active encapsulated treatment composition and instructed to take twice a day orally after a meal and advised to revisit after 5 days.

### 2^{nd} Visit:

The patient revisited 5 days after the previous visit. He obtained some relief but the fever and soreness of throat still persisted. The patient repeated the same medicine, and was advised to revisit after 5 days

### 3^{rd} Visit

The patient re-visited 5 days after the previous visit with great improvement in symptoms. The fever subsided and the sore throat, cough and shortness of breath also subsided. Patient was given the same remedy for an additional 5 days and advised to revisit after 10 days with PCR for COVID-19 report.

### 4^{th} Visit:

The patient revisited 10 days after the previous visit with complete satisfaction. His fever subsided, throat soreness subsided, shortness of breath and cough completely subsided. PCR for COVID-19 report was negative. Patient treatment was stopped.

### Side Effects and Drug Interaction Observed

No serious side effects were observed in any of patients. The side-effects observed, were nausea and vomiting in less than 1% of the patients. One patient developed minor allergic reaction which subsided with treatment with an anti-histamines drug. Drug interactions noted with anti-coagulant drugs. None of the serious adverse effects were observed like, life-threatening conditions such as Steven-Johnsons syndrome or anaphylactic shock with oral therapy.

1. An orally administered pharmacological composition for administration to a patient orally for the treatment of COVID-19 comprising a pharmaceutical carrier and an active composition consisting of about 82% to about 92% Sodium citrate, about 8% to about 18% citric acid and an effective amount of zinc sulfate.

2. An orally administered encapsulated powdered pharmacological composition for administration to a patient orally for the treatment of COVID-19 infections comprising:
a pharmaceutical carrier;
an active composition consisting of an amount of about 82% to about 92% Sodium citrate, about 8% to about 18% citric acid and an effective amount of zinc sulfate, and
wherein the pharmacological composition contains from about 300 mg to about 800 mg of sodium citrate and citric acid and about 10 to 40 mg of zinc sulfate.

3. The composition of claim 2, wherein the active composition includes about 500 mg of sodium citrate and citric acid and about 20 mg zinc sulfate.

4. A safe and effective orally administered pharmacological composition for administration to a patient orally for the treatment of COVID-19 comprising a pharmaceutical carrier and an active composition consisting of about 87% Sodium citrate and about 13% citric acid and an effective amount of zinc sulfate.

5. The composition of claim 2, wherein the active composition consists of about 20 mg zinc sulfate.

6. The composition of claim 1, wherein the orally administered pharmacological composition is an encapsulated pharmacological composition.

7. The composition of claim 2, wherein the orally administered pharmacological composition is an encapsulated powdered pharmacological composition.

8. A method of treating COVID-19 in a patient having such disease, comprising orally administering a therapeutically effective amount of a pharmacological composition comprising:
a pharmaceutical carrier;
an active composition consisting of an amount of about 82% to about 92% Sodium citrate and about 8% to about 18% citric acid, and an effective amount of zinc sulfate,
wherein the pharmacological composition contains from about 300 mg to about 800 mg of Sodium citrate and citric acid and about 10 to 40 mg of zinc sulfate,
wherein the pharmacological composition is safe and effective for the treatment of the COVID-19.

9. The method of claim 2, wherein the active composition includes about 500 mg of sodium citrate and citric acid and about 20 mg zinc sulfate.

10. The method of claim 8, wherein the active composition consists of about 87% Sodium citrate and about 13% citric acid.

11. The method of claim 8, wherein the active composition consists of about 20 mg zinc sulfate.

12. The method of claim 8, wherein the pharmacological composition is taken at least twice a day.
